(19) United States
(12) Patent Application Publication
(10) Pub. No.: US 2024/0325796 A1
(43) Pub. Date: Oct. 3, 2024

(54) Powered Air Filtration Mask with Integrated Illuminators

(71) Applicant: Bailey Lunard Daron Scott, Seattle, WA (US)

(72) Inventor: Bailey Lunard Daron Scott, Seattle, WA (US)

(21) Appl. No.: 18/194,947

(22) Filed: Apr. 3, 2023

(51) Int. Cl. A62B 18/04; A61L 9/20; A62B 7/10; A62B 9/00; A62B 18/08; A62B 23/02

(52) U.S. Cl. CPC A62B 18/045; A61L 9/20; A62B 7/10; A62B 9/00; A62B 18/08; A62B 23/02

(57) ABSTRACT

A powered air filtration mask is a protective face covering device with an integrated filtration and air-handling mechanism that enables active sterilization of both incoming and outgoing volume of air. Aside from the obvious benefits of active sterilization, the constant recirculation of air reduces heat and moisture accumulation for the user. This can improve adherence to rules for proper use of the mask, and thereby increase the overall efficacy of the mask as a tool to reduce pathogenic spread. Further, the device includes a control device that enables the user to easily manipulate and control the electrical components of the present invention. Furthermore, a plurality of illuminators integrated on the device enables the present invention to be used in various outdoor and indoor conditions. Thus, the device is an efficient, multi-functional, comfortable, and user-friendly air filtration mask, that can protect a wearer against airborne pathogens or respiratory hazards.

### FIELD OF THE INVENTION

The present invention relates generally to a personal protective equipment configured to protect a wearer against airborne pathogens or respiratory hazards. More specifically, the present invention relates to a face mask with an integrated filtration and air-handling mechanism.

### BACKGROUND OF THE INVENTION

The current and foreseeable hazards presented by the ongoing COVID-19 pandemic has highlighted a demand for effective, reusable personal protective gear. In particular, the effectiveness of conventional cloth and polyester masks in limiting the spread of airborne pathogens is cited as an area for improvement. Passive filtration and droplet-capture does provide a reduction in pathogen spread, but it is proposed that active sterilization of incoming and outgoing volume of air, coupled with a forced recirculation of the air inside the mask, will improve the efficacy of this style of protective wear. However, a protective face covering that can perform active sterilization is a rare find in the current market.

An objective of the present invention is to provide users with a protective face covering device that can perform multiple functionalities. To that end, the present invention is a powered face covering device with an integrated filtration and air-handling mechanism, that enables active sterilization of both incoming and outgoing volume of air. Aside from the obvious benefits of active sterilization, the constant recirculation of air by the present invention reduces heat and moisture accumulation for the user. This can improve adherence to rules for proper use of the mask, and thereby increase the overall efficacy of the masks as a tool to reduce pathogenic spread. Further, the present invention comprises other components such as a control device, plurality of illuminators, etc. that are powered and enables the present invention to be used in various outdoor and indoor conditions. Thus, the present invention is an efficient, comfortable, and user-friendly air filtration mask, that can protect a wearer against airborne pathogens or respiratory hazards.

### SUMMARY OF THE INVENTION

A powered air filtration mask is a protective face covering device with an integrated filtration and air-handling mechanism that enables active sterilization of both incoming and outgoing volume of air. Aside from the obvious benefits of active sterilization, the constant recirculation of air by the present invention reduces heat and moisture accumulation for the user. This can improve adherence to rules for proper use of the mask and thereby increase the overall efficacy of the masks as a tool to reduce pathogenic spread. Further, the present invention comprises a control device that enables the user to easily manipulate and control the electrical components of the present invention. Furthermore, a plurality of illuminators integrated on the present invention enables the present invention to be used in various outdoor and indoor conditions. Thus, the present invention is an efficient, multi-functional, comfortable, and user-friendly air filtration mask, that can protect a wearer against airborne pathogens or respiratory hazards.

### BRIEF DESCRIPTION OF THE DRAWINGS

FIG. 1 is a top-front-left perspective view of the present invention.

FIG. 2 is a top-rear-right perspective view of the present invention.

FIG. 3 is an exploded perspective view of the present invention.

FIG. 4 is a front elevational view of the present invention.

FIG. 5 is a rear elevational view of the present invention.

FIG. 6 is a left-side elevational view of the present invention.

FIG. 7 is a magnified view taken about circle 7 in FIG. 6.

### DETAIL DESCRIPTIONS OF THE INVENTION

All illustrations of the drawings are for the purpose of describing selected versions of the present invention and are not intended to limit the scope of the present invention.

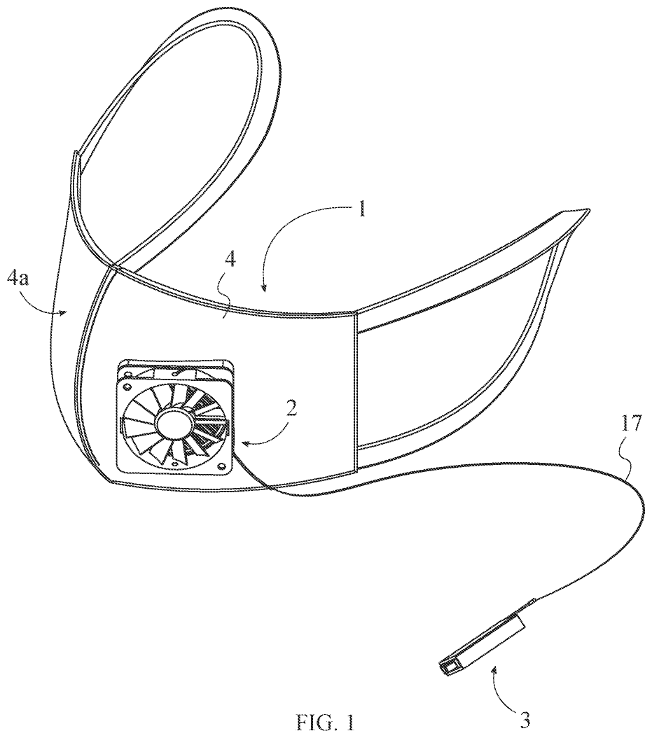

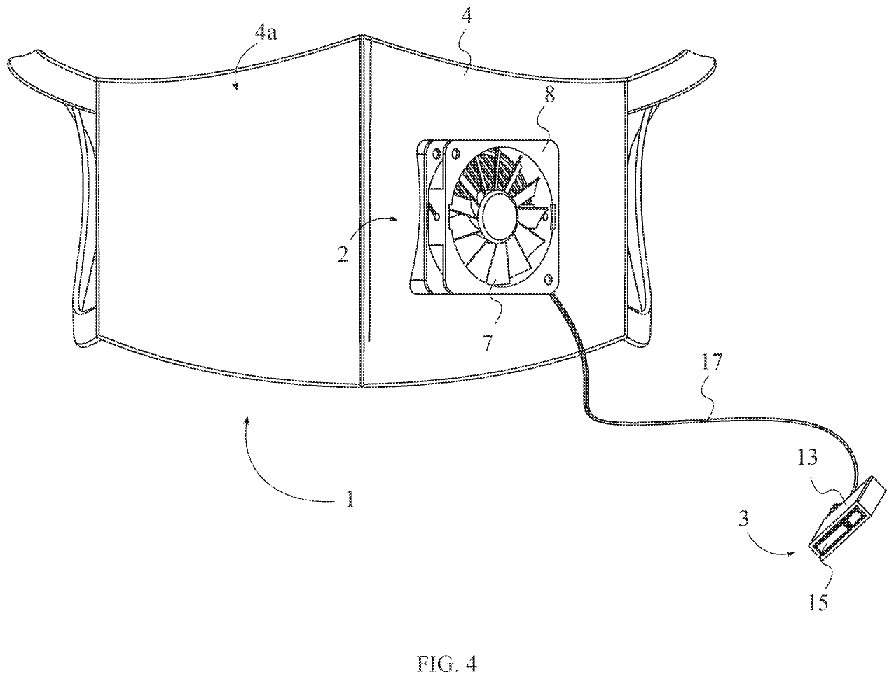

The following description is in reference to FIG. 1 through FIG. 7. According to a preferred embodiment, the present invention comprises a facial covering 1, a filtration module 2, and at least one controller device 3. The facial covering 1 comprises a covering fabric 4, an aperture 5, and at least one support structure 6. Preferably, the facial covering 1 may be adapted from conventional facemasks or surgical coverings by forming the aperture into the covering fabric 4 of the facial covering. The covering fabric 4 may comprise one or more layers of any skin-friendly fabric that is comfortable to use over a user's face. Further, the covering fabric 4 comprises a first surface 4a and a second surface 4b, wherein the first surface 4a is positioned opposite to the second surface 4b across the covering fabric 4. Preferably, the first surface 4a constitutes an exterior surface of the covering fabric 4 and the second surface 4b constitutes an interior surface of the covering fabric 4 that comes in contact with the user's face. Further, the covering fabric 4 and the facial covering 1 may comprise any material, shape, size, components, arrangement of components, etc. that are known to one of ordinary skill in the art, as long as the intents of the present invention are not altered. For example, the facial covering 1 may comprise loops, strings, etc. or any other fastening means for attaching the covering fabric 4 to the user's face.

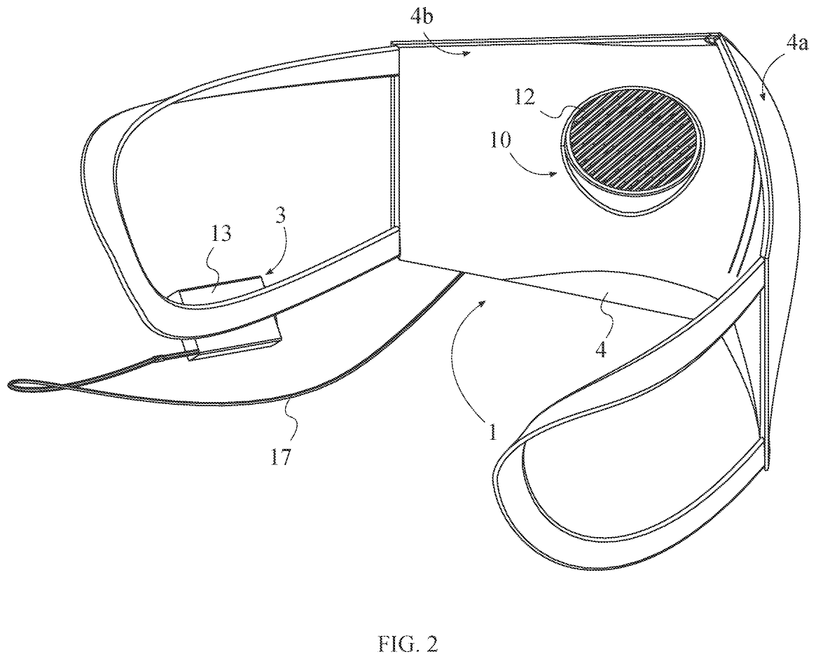

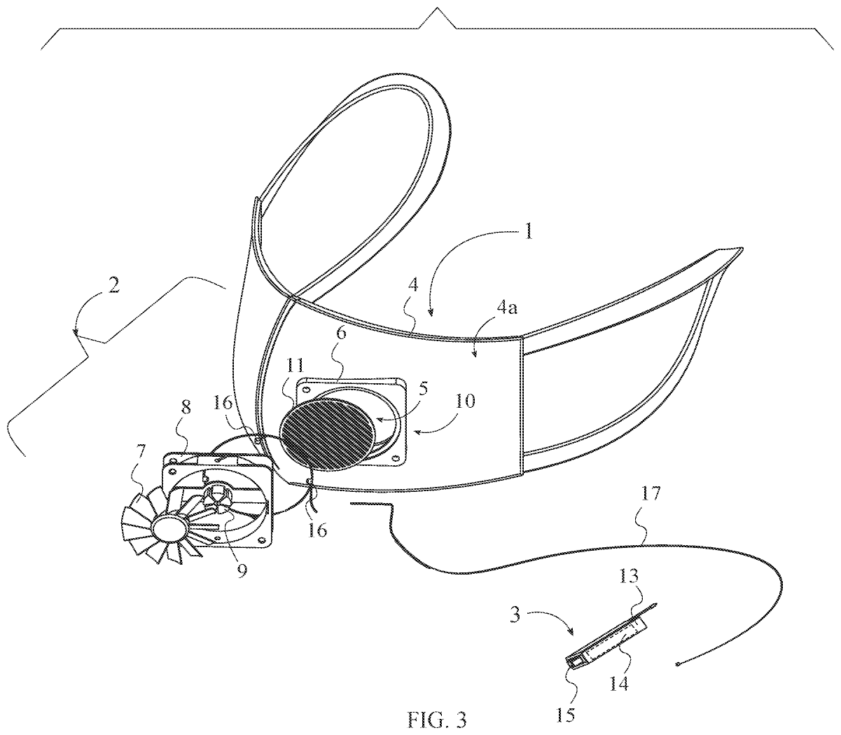

In the preferred embodiment, the aperture 5 traverses through the covering fabric 4, and the support structure 6 is mounted within the aperture 5. In other words, the aperture 5 has the supporting structure 6 positioned across the internal and external surfaces of the covering fabric 4 as shown in FIG. 2 and FIG. 3, wherein said supporting structure 6 is positioned into the aperture 5 to maintain a uniform aperture profile congruent to the output of the filtration module 2. In other words, the support structure is positioned across the first surface 4a and the second surface 4b of the covering fabric 4. Preferably, the aperture 5 is a hole or opening that travers through the covering fabric 4, and the support structure 6 is a sturdy yet light-weight structure, preferably made of hard plastic, that can house the electrical and filtration components of the present invention in a safe and compact fashion. As seen in FIG. 3, the aperture 5 is a circular hole passing through the covering fabric 4, and the support structure 6 is a rectangular frame attached along the perimeter of the aperture 5. The support structure 6 may be attached to the covering fabric 4 through any fastening technique that is known to one of ordinary skill in the art, such as through sewing, using adhesives, clips, etc. Further, the aperture 5 and the support structure 6 may comprise any shape, size, components, arrangement of components, etc., as long as the objectives of the present invention are not altered.

Continuing with the preferred embodiment, the filtration module 2 is integrated into the facial covering 1, wherein the filtration module 2 is mounted within and onto the support structure 6. The filtration module 2 constitutes a powered air purification device, containing and supporting all essential mechanisms for pressurizing and sanitizing a volume of air. The filtration module 2 may be configured bimodally, wherein the affected flow direction of the volume of air may be directed into the facial covering 1 to overpressure the internal cavity of the facial covering 1 or may be configured to exhaust a user's breath from within the facial covering 1. To accomplish this, the filtration module 2 comprises an impeller 7, a duct housing 8, at least one motor 9, and a filter element 10. Preferably, the impeller 7 is a fan blade assembly configured for bidirectional air handling. More specifically, the impeller 7 is suitable for moving air coaxial to the impeller 7 in either axial direction dependent on the direction of rotation for the impeller 7. The duct housing 8 is a sturdy frame that holds the impeller 7 and the motor 9. Accordingly, the impeller 7 and the motor 9 are concentrically mounted within the duct housing 8. In the preferred embodiment, the motor 9 is mounted concentric to the impeller 7 to impart rotational motion of the impeller 7 relative to the duct housing 8. In other words, the impeller is operatively coupled to the motor 9, wherein the motor 9 is used to drive a rotation of the impeller, wherein the rotation of the impeller 7 within the duct housing 8 forces a volume of air to traverse the duct housing 8 between the interior and the exterior of the facial covering 1. The forced recirculation of air within the facial covering 1 ideally alleviates any moisture or heat buildup within the facial covering 1, thereby reducing user discomfort during extended use. Consequently, the present invention is more likely to prevent pathogenic spread when compared to conventional facemasks by encouraging users to wear the facial covering properly, i.e., continuously while indoors or enclosed spaces.

In the preferred embodiment, the duct housing 8 is laterally mounted onto the support structure 6, and the impeller 7 is laterally offset from the filter element 10 within the support structure 6. This is so that air from outside may be pulled in by the impeller 7 towards the filter element 10, or humid air from within the facial covering 1 may be pushed out through the aperture 5 and the impeller 7. More specifically, the filter element 10 is in fluid communication with the impeller 7 through the aperture 5. To enable this, the impeller 7 is positioned laterally offset from the first surface 4a of the covering fabric 4 and the filter element 10 is positioned adjacent to the first surface 4a of the covering fabric 4.

Continuing with the preferred embodiment, the controller device 3 is electrically connected to the motor 9, and the controller device 3 is operably coupled to the impeller 7 through the motor 9, wherein operating the controller device 3 governs actuation of the impeller 7. More specifically, the controller device 3 constitutes an interface device and input mechanism by which a user may moderate and select the operating conditions for the at least one motor 9. It should be noted that the controller device 3, the motor 9, and the impeller 7 may comprise any brand, size, shape, components, arrangement of components, etc. that are known to one of ordinary skill in the art, as long as the intents of the present invention are not altered.

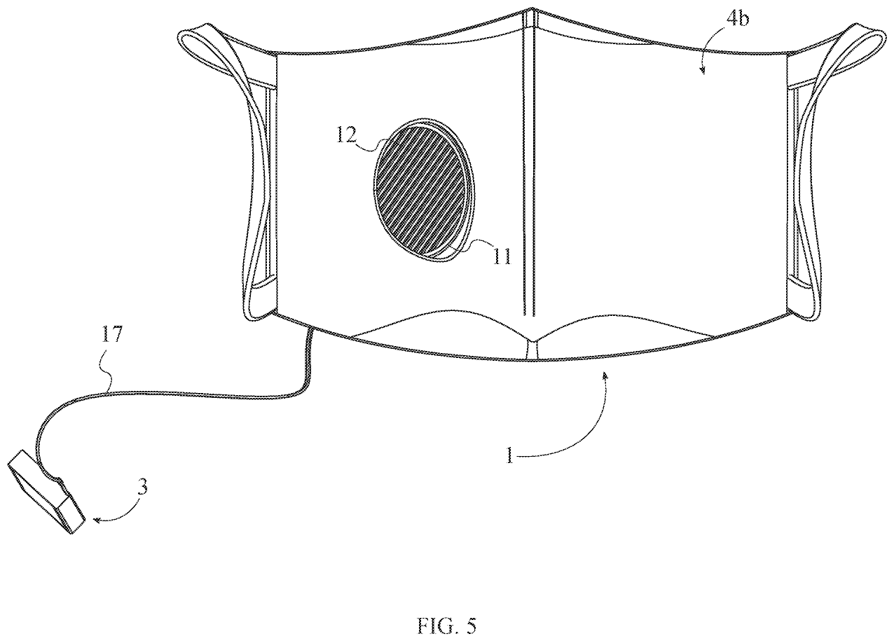

A more detailed description of the present invention follows. In some embodiments, the filter element 10 comprises a filter frame 11 and a filtering media 12. As shown in FIG. 3 and FIG. 5, the filter element 10 generally defines a static membrane or particulate-based filtration component inserted into the support structure 6 in fluid communication with the impeller 7 through the aperture 5. The position of the filter element 10 substantially occluding the aperture 5 forces the volume of air affected by the impeller 7 across the filter element 10 before being inhaled by a user, or prior to being exhausted from the facial covering 1. Accordingly, the filter frame 11 is perimetrically mounted within the support structure 6, and the filtering media 12 is releasably engaged within the filter frame 11. This arrangement enables a user to exchange the filtering media 12 from within the frame as the filter media becomes saturated or otherwise expended from prolonged exposure to contaminants. It should be noted that, the filter frame 11 and the filtering media 12 may comprise any material, size, shape, etc. as long as the intents of the present invention are not altered.

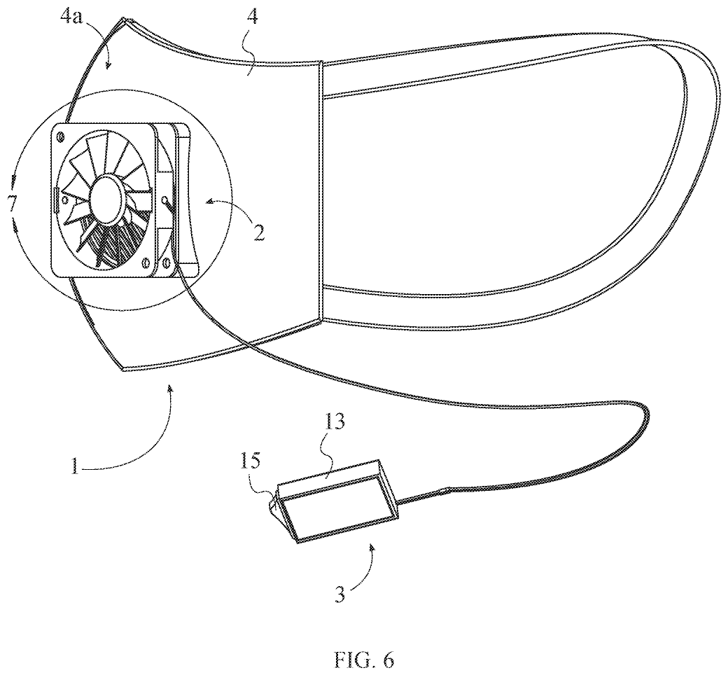

The operation of the at least one motor 9 is moderated by at least one controller device 3 electrically connected through the duct housing 8 as shown in FIG. 1 and FIG. 6. As previously mentioned, the controller device 3 constitutes an interface device and input mechanism by which a user may moderate and select the operating conditions for the at least one motor 9. Accordingly, in the preferred embodiment, the controller device 3 comprises a receptacle 13, a power supply 14, and a control switch 15. Preferably, the receptacle 13 is a rectangular housing within which the electrical components are housed and/or connected. As seen in FIG. 3, the power supply 14 is mounted within the receptacle 13, and the control switch 15 is laterally mounted onto the receptacle 13. In the preferred embodiment, the power supply 14 is a cellular battery pack, and the control switch 15 is a momentary switch configured to moderate the flow of electrical power between the power supply 14 and the motor 9. To that end, the power supply 14 is electrically connected to the motor 9 through the control switch 15. Further, the control switch 15 may be configured to reverse the electrical current to reverse the rotation of the motor 9 and, by extension, the impeller 7. By actively moderating the flow direction, a user may approximate a normal respiratory pattern of intake and exhaust functions coinciding with the inhalations and exhalations of the user. The receptacle 13, the control switch 15 and the power supply 14 may comprise any size, shape, technology, components, arrangement of components, orientation, location etc. as long as the intents of the present invention are not altered.

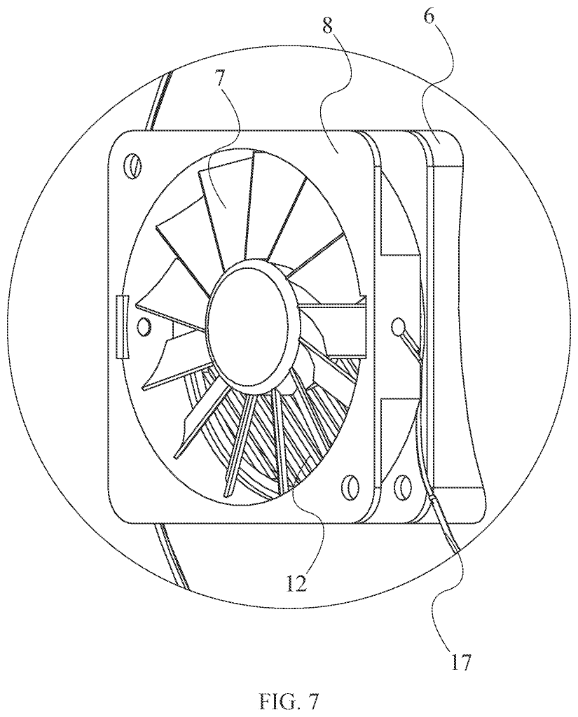

Continuing with the preferred embodiment, the present invention comprises a plurality of illuminating devices 16. As seen in FIG. 3 and FIG. 7, the plurality of illuminating devices 16 is radially distributed around the duct housing 8. The plurality of illumination devices 16 are ideally light-emitting diode (LED) assemblies configured to output a variety of selectable colors and ornamental effects. Preferably, the plurality of illuminating devices 16 is electrically connected to the controller device 3 so as to provide operating electrical current to the plurality of illumination devices 16 as configured by a user. Accordingly, the controller device 3 is operably coupled to the plurality of illuminating devices 16, wherein operating the controller device 3 governs controlling and activation of the plurality of illuminating devices 16. More specifically, the control device 3 moderates and activates the plurality of illumination devices 16 according to preset values stored within the controller device 3. In one instance, an activation pattern for the plurality of illumination devices 16 corresponds to any period between exhalation and inhalation cycles of the motor to create a visual effect mimicking normal breathing patterns. The plurality of illumination devices 16 may also be configured to shift through any variety or combination of visible colors to present unique ornamental displays in conjunction with the rotating impeller 7.

In another embodiment, the plurality of illumination devices 16 may be configured to emit ultraviolet radiation into the duct housing 8, specifically a germicidal volume of UV-C radiation in fractions of suitable potency to sterilize any volume of air passing therethrough. The exposure duration and potency may be varied according to any mode of use or rate of airflow, corresponding to the requisite fraction of ultraviolet radiation for any notable antimicrobial effects to be rendered onto the volume of air traversing the duct housing.

Continuing with the preferred embodiment, the present invention comprises at least one connecting wire 17. As seen in FIG. 1 through FIG. 6, the connecting wire 17 is positioned between the controller device 3 and the filtration module 2, wherein the connecting wire 17 electrically connects the controller device 3 to the filtration module 2. Preferably, the connecting wire 17 is an insulated metal wire that can conduct electricity. However, any other connection mechanism may be used for controlling the filtration module 2, as long as the objectives of the present invention are fulfilled. Examples of connection mechanisms include, but are not limited to remote control using wireless communication modules, voice activation techniques, sensor-controlled activation techniques, etc.

Although the invention has been explained in relation to its preferred embodiment, it is to be understood that many other possible modifications and variations can be made without departing from the spirit and scope of the invention as hereinafter claimed.

What is claimed is:

1. A powered air filtration mask device comprising:
a facial covering;
a filtration module;
at least one controller device;
the facial covering comprising a covering fabric, an aperture, and at least one support structure;
the filtration module comprising an impeller, a duct housing, at least one motor, and a filter element;
the covering fabric comprising a first surface and a second surface;
the first surface being positioned opposite to the second surface about the covering fabric;
the aperture traversing through the covering fabric;
the support structure being mounted within the aperture;
the filtration module being integrated into the facial covering;
the filtration module being mounted within the support structure;
the duct housing being laterally mounted onto the support structure;
the impeller and the motor being mounted within the duct housing;
the impeller being operatively coupled to the motor, wherein the motor is used to drive a rotation of the impeller;
the motor and the impeller being laterally offset from the filter element within the support structure;
the filter element being in fluid communication with the impeller through the aperture; and
the controller device being electrically connected to the motor.

2. The powered air filtration mask device of claim 1 further comprising:
the filter element comprising a filter frame and a filtering media;
the filter frame being perimetrically mounted within the support structure; and
the filtering media being releasably engaged within the filter frame.

3. The powered air filtration mask device of claim 1 further comprising:
the controller device comprising a receptacle, a power supply and a control switch;
the power supply being mounted within the receptacle;
the control switch being laterally mounted onto the receptacle; and
the power supply being electrically connected to the motor through the control switch.

4. The powered air filtration mask device of claim 1 further comprising:
a plurality of illuminating devices;
the plurality of illuminating devices being radially distributed around the duct housing; and
the plurality of illuminating devices being electrically connected to the controller device.

5. The powered air filtration mask device of claim 4, wherein the plurality of illuminating device are LED (light emitting diode) assemblies.

6. The powered air filtration mask device of claim 4, wherein the plurality of illuminating devices is configured to emit ultraviolet radiation into the duct housing.

7. The powered air filtration mask device of claim 1, wherein the impeller is positioned laterally offset from the first surface of the covering fabric and the filter element is positioned adjacent to the first surface of the covering fabric.

8. The powered air filtration mask device of claim 1 further comprising:
at least one connecting wire;
the connecting wire being positioned between the controller device and the filtration module; and
the connecting wire electrically connecting the controller device to the filtration module.

9. The powered air filtration mask device of claim 1, wherein the support structure is positioned across the first surface and the second surface of the covering fabric.

10. The powered air filtration mask device of claim 1, wherein the impeller is a fan blade assembly configured for bidirectional air handling.

11. The powered air filtration mask device of claim 1, wherein rotation of the impeller within the duct housing forces a volume of air to traverse the duct housing between the first surface and the second surface of the facial covering.

12. The powered air filtration mask device of claim 1, wherein the motor is mounted concentric to the impeller.

13. A powered air filtration mask device comprising:
a facial covering;
a filtration module;
at least one controller device;
the facial covering comprising a covering fabric, an aperture, and at least one support structure;
the filtration module comprising an impeller, a duct housing, at least one motor, and a filter element;
the controller device comprising a receptacle, a power supply and a control switch;
the covering fabric comprising a first surface and a second surface;
the first surface being positioned opposite to the second surface about the covering fabric;
the aperture traversing through the covering fabric;
the support structure being mounted within the aperture;
the filtration module being integrated into the facial covering;
the filtration module being mounted within the support structure;
the duct housing being laterally mounted onto the support structure;
the impeller and the motor being mounted within the duct housing;
the impeller being operatively coupled to the motor, wherein the motor is used to drive a rotation of the impeller;
the motor and the impeller being laterally offset from the filter element within the support structure;
the filter element being in fluid communication with the impeller through the aperture, wherein the impeller is positioned along the first surface of the covering fabric and the filter element is positioned along the second surface of the covering fabric;
the power supply being mounted within the receptacle;
the control switch being laterally mounted onto the receptacle; and
the power supply being electrically connected to the motor through the control switch.

14. The powered air filtration mask device of claim 13 further comprising:
the filter element comprising a filter frame and a filtering media;
the filter frame being perimetrically mounted within the support structure; and
the filtering media being releasably engaged within the filter frame.

15. The powered air filtration mask device of claim 13 further comprising:
a plurality of illuminating devices;
the plurality of illuminating devices being radially distributed around the duct housing; and
the plurality of illuminating devices being electrically connected to the controller device.

16. The powered air filtration mask device of claim 15, wherein the plurality of illuminating device are LED (plurality of light emitting diode) assemblies.

17. The powered air filtration mask device of claim 15, wherein the plurality of illuminating devices is configured to emit ultraviolet radiation into the duct housing.

18. The powered air filtration mask device of claim 13 further comprising:
at least one connecting wire;
the connecting wire being positioned between the controller device and the filtration module; and
the connecting wire electrically connecting the controller device to the filtration module.

19. The powered air filtration mask device of claim 13, wherein the impeller is a fan blade assembly configured for bidirectional air handling.

20. The powered air filtration mask device of claim 13, wherein the motor is mounted concentric to the impeller.
